(19) United States
(12) Plant Patent Application Publication
(10) Pub. No.: US 2020/0113108 P1
(43) Pub. Date: Apr. 9, 2020

(54) Calla lily plant named 'Diva Himalaya'

(50) ;

(71) Applicant: Dummen Group B.V., De Lier (NL)

(72) Inventor: Peter Beckman, Santa Cruz, CA (US)

(21) Appl. No.: 16/602,427

(22) Filed: Oct. 2, 2019

Related U.S. Application Data
(60) Provisional application No. 62/766,213, filed on Oct. 5, 2018.

(51) Int. Cl. A01H 6/56

(52) U.S. Cl. PLT/490

(57) ABSTRACT

‘Diva Himalaya’ is a new variety of calla lily plant having white colored spathes and thin, strong peduncles. The leaves are green with no maculations present.

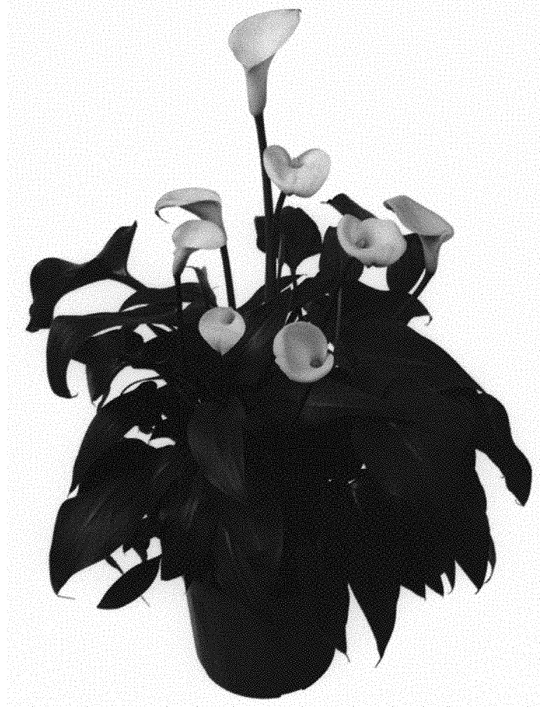

### CROSS REFERENCE TO RELATED APPLICATION

This application claims the benefit of U.S. Provisional Patent Application Ser. No. 62/766,213, having a filing date of Oct. 5, 2018, and entitled “Varieties of Z. aethiopica Calla Lily Plants”, the entire disclosure of which is incorporated herein by reference.

Botanical classification: Zantedeschia aethiopica.

Varietal denomination: ‘Diva Himalaya’.

### BACKGROUND OF THE NEW PLANT

The present invention comprises a new and distinct cultivar of calla lily, botanically known as Zantedeschia aethiopica and hereinafter referred to by the cultivar name ‘Diva Himalaya’.

‘Diva Himalaya’ is a product of a planned breeding program and the new cultivar was selected in Santa Cruz, Calif. from the F2 population of the crossing of unpatented Zantedeschia seedling 7043-BK (female parent) with unpatented Zantedeschia seedling 94109-56 (male parent).

Asexual reproductions of ‘Diva Himalaya’ have demonstrated that the combination of characteristics as herein disclosed for the new variety are retained and reproduced true to type through successive generations of asexual reproduction.

Color references are made to The 2001 R.H.S. Colour Chart of The Royal Horticultural Society of London, 4^{th} Edition, except where general color terms of ordinary significance are used.

The present invention has not been evaluated under all possible environmental conditions. The phenotype may vary with variations in environment without a change in the genotype of the plant.

The following traits have been repeatedly observed and determined to be basic characteristics of ‘Diva Himalaya’ which, in combination, distinguish this calla lily as a new and distinct cultivar:

1. White colored spathes;

2. No leaf maculations;

3. Early production time;

4. Thin, strong peduncles; and

5. Easy to transport.

Table 1 provides a comparison of ‘Diva Himalaya’ to similar Zantedeschia aethiopica varieties ‘Avalanche’ (unpatented) and ‘Innocence’ (unpatented).

TABLE 1

| Comparison | Differing | Comparison | Present variety |
|---|---|---|---|
| variety | trait(s) | expression | expression |
| ‘Avalanche’ | Leaf maculations | Present | None present |
| ‘Innocence’ | Spathe color | White Group | White Group |
| | | RHS 155B | RHS 155D |

### BRIEF DESCRIPTION OF THE DRAWING

The accompanying photographic drawing illustrates the new variety, with the colors being as nearly true as is possible with color illustrations of this type.

### DESCRIPTION OF THE NEW PLANT

Type: Evergreen.
Main use: Potted plant and cut flower.
Production time: Early, after 18-24 weeks.
Disease/pest resistance: Very strong tolerance to Pythium root rot and bacteria soft rot.
Leaves: Green, with no maculations present.
Peduncles: Thin and strong.
Transport: Easy.
Spathe:
   Length.—9-12 cm.
   Width.—6.5-8 cm.
   Throat spot.—Absent.
   Color.—White Group RHS 155D.

I claim:

1. A new and distinct variety of calla lily plant named ‘Diva Himalaya’ as herein described and illustrated.
